(19) United States
(12) Patent Application Publication
(10) Pub. No.: US 2014/0238400 A1
(43) Pub. Date: Aug. 28, 2014

(54) RESPIRATORY MASK

(71) Applicant: INTERSURGICAL AG, Vaduz (LI)

(72) Inventor: Andrew Neil Miller, Wokingham (GB)

(73) Assignee: INTERSURGICAL AG, Vaduz (LI)

(21) Appl. No.: 13/775,985

(22) Filed: Feb. 25, 2013

(51) Int. Cl. A61M 16/06; B29C 45/16; A61M 16/00

(52) U.S. Cl. CPC A61M 16/06; A61M 16/0057; B29C 45/1676
USPC 128/205.25; 128/206.28; 128/206.24; 264/254

(57) ABSTRACT

This invention concerns a respiratory mask for delivering inspiratory gas to a wearer. The respiratory mask has a mask body of generally concave shape and having a peripheral edge. The mask body being is formed of a first material and adapted to provide a cavity in use about the mouth and nose of a wearer such that the inhalation gas can be inhaled by the patient from the cavity. The mask body has an inlet port that is engageable with a supply of inhalation gas and in communication with the cavity. The mask body has a resilient seal formation depending from at least a portion of the peripheral edge. The mask body has at least one panel formed of a second material that is softer than the first material of the mask body so as to allow lateral deflection of the mask body to accommodate different facial widths.

### FIELD OF THE INVENTION

The present invention relates to respiratory masks suitable for delivery of gas for inhalation by a patient and more particularly, although not exclusively, to a mask which is suitable for use in therapy.

### BACKGROUND OF THE INVENTION

Respiratory masks are used to supply inhalation gases, and possibly also atomised liquids such as drugs in solution, to the airways of a patient. In general, a gas is supplied to a respiratory enclosure defined by the respiratory mask and the face of the patient, and the patient inhales the inhalation gas from this respiratory enclosure. Conventional masks typically also have an inlet for the inhalation gas, and an outlet through which exhaled gas escapes the respiratory mask.

UK patent GB 2 412 594, in the name of Intersurgical Limited, discloses an example of such a respiratory mask. Masks of this type used in the therapy of patients can be distinguished from other breathing equipment which may be used by divers or members of the emergency services. Industrial breathing sets of that kind are generally complex and expensive and intended to provide a reusable source of breathable air to the user in inhospitable environments. In contrast, respiratory masks used for the therapy of patients, for example, within medical facilities are intended to be lightweight, typically disposable, articles merely for the delivery of gases to a patient, rather than protection from the environment.

Conventional respiratory masks for patient therapy typically comprise a unitary component defining a cavity and an outwardly-turned peripheral rim that is urged against the wearer's face, about their nose and mouth. The unitary component is typically a relatively thin-walled plastic structure, e.g. formed of polyvinylchloride (PVC), such that it provides a defined cavity shape, whilst offering a degree of flexibility to ensure a good fit against the wearer.

In order to maintain an effective seal between the peripheral rim of the respiratory mask and the patient's face, conventional respiratory masks have an elasticated strap that is placed about the patient's head so as to urge the respiratory mask against the face of the patient. For some conventional masks, a nose clip may be fitted about the portion of the mask that surrounds the patient's nose so as to further improve the seal between the mask and the patient's face.

The relatively flexible PVC material of conventional respiratory masks allows these masks to conform to the contours of a patient's face to a limited degree in order to provide a seal. However it has been found that the quality of the seal can vary significantly for wearer's of differing facial dimensions or profiles such that air may be entrained around the edge of the mask in use. Additionally gas, intended for inhalation, may be leaked around the mask edges. Any such leakage requires the delivery of a larger volume of gas and/or an elevated delivery pressure to a patient. Leakage of certain gases may be detrimental to others in the vicinity of the patient, such as carers or visitors. An insufficient seal also leads to uncertainty over the concentration of fluid being inhaled by the patient.

It is therefore desirable to provide a mask which is formed substantially of a more rigid plastics material but which has a seal about the periphery of the mask which is formed of a more supple material. In attempting to provide an improved seal, it is generally considered necessary to provide a tighter fit to the patient's face. This can be achieved by tightening the strap which passes around the patient's head during use. However it has been found that a tighter fit to a patient's face generally implies a more limited range of fit that can be accommodated by a mask.

It is an aim of the present invention to provide a respiratory mask which can accommodate a range of face shapes and sizes whilst providing an adequate seal.

### BRIEF SUMMARY OF THE INVENTION

According to the invention, there is provided a respiratory mask for delivering inspiratory gas to a wearer, the respiratory mask comprising a mask body of generally concave shape and having a peripheral edge, the mask body being formed of a first material and adapted to provide a cavity in use about the mouth and nose of a wearer such that the inhalation gas can be inhaled by the patient from the cavity, wherein the mask body has at least one panel formed of a second material that is softer than the first material.

The mask body may have an inlet port that is engageable with a supply of inhalation gas and in communication with the cavity. The mask body may have a resilient seal formation depending from at least a portion of the peripheral edge,

The mask may have first, or upper, and second, or lower, ends. The mask body may be shaped to accommodate the nose of a wearer towards the first end of the mask and to accommodate the mouth of a wearer towards the second end of the mask. Accordingly the mask body may have respective nose and mouth portions.

The panels provide the advantage of allowing the mask body to deform sideways or laterally (e.g. relative to a direction between the first and second ends of the mask) to accommodate varying facial widths or profiles. The panels allow the mask to stretch in a manner akin to more-conventional PVC masks whilst offering the benefits of an alternative material construction and improved edge seal.

The panels may be formed of an elastomeric material. The panels may be elongate in nature. The panels may be disposed on the mouth portion of the mask body. The panels may extend in a direction between the first and second ends. The panels may extend between the mouth and the nose portions of the mask body. The panels and the seal formation may be formed of the same elastomeric material. The elastomeric material is preferably a thermoplastic elastomer.

The resilient seal formation may comprise both an inwardly and outwardly depending lip portion relative to the peripheral edge of the body. The resilient seal formation may be elastomeric. This may serve to improve the seal formed between the mask body and the face of the patient, and may also provide a more comfortable fit for a patient. The seal portion may have discontinuities therein in the form of slits which allow the seal to deform about the different contour portions of a wearer's face

A relatively rigid mask body (i.e. the first material) preferably defines the shape of the cavity and may be considered to provide an outer cavity wall. The mask body material may provide a support structure for the second, softer material. By “rigid” mask body is meant that the mask body substantially maintains its shape when subjected to normal handling conditions. The mask body is preferably formed of a plastic material, typically in an injection moulding process. Most preferably, the mask body is formed of polypropylene.

The mouth portion of the mask body may comprise a forward-facing front wall and laterally protruding sidewalls. The nose portion of the mask body may comprise laterally protruding side walls depending rearwardly from an apical or tip portion of the nose portion and/or mask body.

The seal formation and flexible panels may also be formed by injection moulding. The body, seal, and/or panels may be co-formed by way of a moulding process. The body, seal, and/or panels may be co-formed as part of a so-called multi-shot injection moulding process. The first and second material parts of the respiratory mask may be bonded together by this process.

The inlet port preferably comprises an opening in the wall of the cavity, and a conventional tubular connector extending outwardly therefrom. Most preferably the tubular connector extends from an opening in the nose portion of the mask body into a space adjacent to the mouth portion. The respiratory mask body may include exhalation openings that allow exhaled gases to escape from the cavity of the respiratory mask, during use. When the respiratory mask is to be used for delivering a high concentration of an inhalation gas, such as oxygen, to a patient, the openings in the mask body may each include a valve. The openings may be placed at a lower end of the flexible panels.

The mask body may have one or more openings therein, said one or more opening being at least partially filled with the second material. The opening may be only partially filled with the second material such that the unfilled portion defines an outlet opening in the mask body.

The second material portion, i.e. the panel, may be substantially flush with the first material portion of the mask body. The mask body and panel may have substantially equal wall thickness. The panels may be substantially planar or flat in form.

The mask body may have outwardly extending flange formations located on either side. The flange formations may be adapted to receive an elasticated cord or strap. The elasticated cord or strap may be formed of elastomeric material, and may therefore be formed integrally with the remainder of the mask using the injection moulding process. Alternatively, the elasticated cord or strap may be formed as a separate component.

The mask may be a patient therapy mask, such as an oxygen or aerosol therapy mask.

### BRIEF DESCRIPTION OF THE DRAWINGS

Practicable embodiments of the invention are described in further detail below by way of example only with reference to the accompanying drawings, of which:

FIG. 1 shows a front view of a mask according to an example of the invention;

FIG. 2 shows a side view of the mask of FIG. 1;

FIG. 3 shows a front view of a mask according to a second example of the present invention; and,

FIG. 4 shows a side view of the mask of FIG. 3.

### DETAILED DESCRIPTION OF THE INVENTION

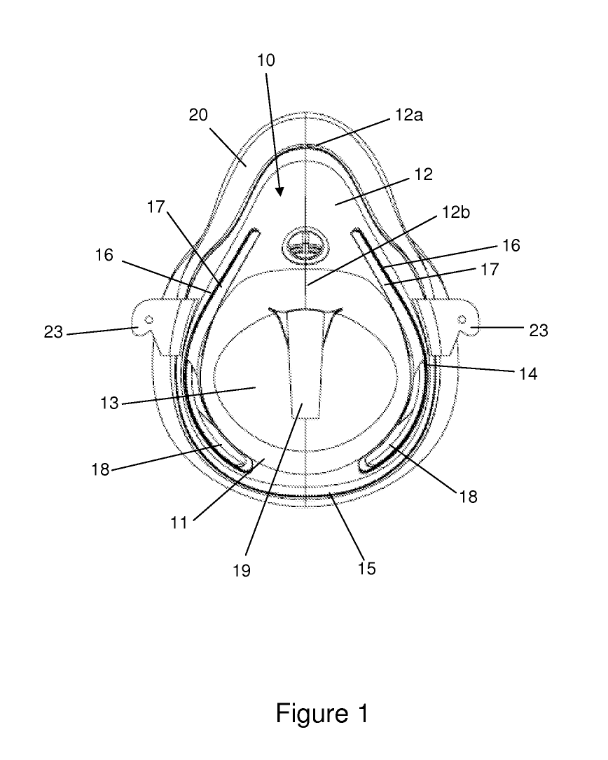

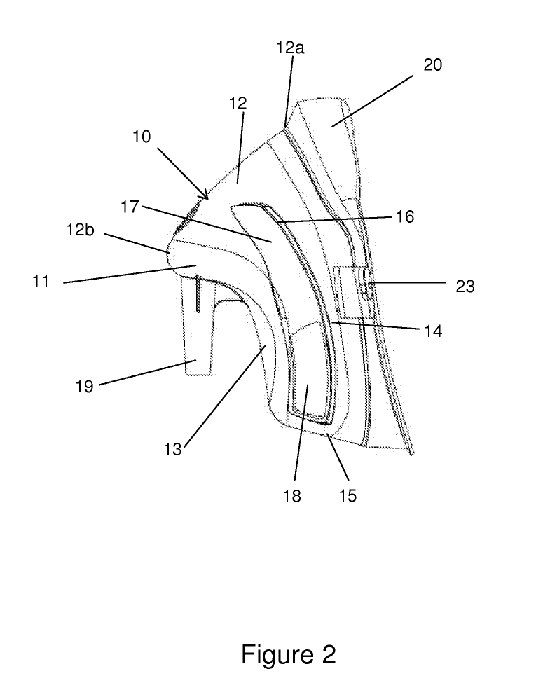

Turning firstly to FIGS. 1 and 2, there is shown a respiratory mask, which is suitable for the delivery of respiratory gases, such as oxygen, to a wearer, such as a patient. The respiratory mask 10 is particularly suited to patient therapy and may be used for example within a medical facility, such as a hospital.

The respiratory mask comprises a mask body 10, formed from a suitably strong and relatively rigid plastics material, such as polypropylene or polyvinyl chloride, and one or more relatively flexible or complaint material (i.e. a softer material), such as an elastomer. The harder and softer materials are used to form different portions of the mask as will be described below. A Styrene-Ethylene-Butylene-Styrene (SEBS)-based thermoplastic elastomer may be used for the softer material. However it will be appreciated that other thermoplastic elastomers or alternative conventional mask body and seal materials may be used. Any such material will typically have a Shore A hardness of less than 90, 80 or 70.

The softer, elastomer material is used in this embodiment to form a sealing formation 20 about the periphery of the mask body 10.

The respiratory mask is manufactured using a so-called two-shot, or multi-shot, injection moulding process. In particular, the mask body 10 is firstly injection moulded as a single component, and the sealing formation 20 is then injection moulded onto the mask body 10. The mask body 10 and the sealing formation 20 are bonded together by this process.

The mask body 10 is generally concave, so as to define a cavity via which an inhalation gas is delivered to a patient, and comprises a mouth portion 11 and a nose portion 12. The mask body 10 is shaped such that the maximum depth of the cavity defined by the nose portion 12 is greater than the depth of the cavity defined by the mouth portion 11. The nose portion 12 is generally tapered towards a first end of the mask 12a that is shaped to fit around the bridge of the patient's nose. The nose portion 12 also tapers forwardly towards a tip or apex 12b which is the forward-most part of the mask body.

The mouth portion 11 generally comprises a forward-facing front wall 13 and laterally-protruding side wall portions 14, which are arranged to be located adjacent a wearer's cheeks or jowls, and particularly the lower portion thereof, in use. The mouth portion 11 also comprises a lower wall 15 or sill formation beneath the front wall 13, i.e. at a second end of the mask, which is intended to contact with a wearer's chin.

The side wall portions 14 may be considered to span the mouth 11 and nose 12 portions such that the side walls 14 extend up either side of the mask body 10 as a whole. Such side wall portions 14 extend laterally or outwardly towards the peripheral edge of the mask on each side thereof.

The side wall portions 14 are formed of the rigid mask body material and each have an elongate opening 16 therein extending in a direction from the first end 12a towards the opposing end (i.e. lower end) of the mask body provided at lower wall 15. The elongate openings 16 are substantially symmetrically arranged on either side of a central longitudinal axis of the mask body. The elongate openings 16 are typically greater than half the length of the mask body and, in this example, may be greater than or approximately to two-thirds or three-quarters of the length of the mask body 10. As can be seen in FIG. 2, the openings 16 may be generally quadrilateral, rectangular or trapezoidal in shape except that the long sides thereof follow the curvature or profile of the mask body 10 in which they are formed.

The openings 16 are partially filled with a material that is more compliant and flexible than that of the mask body 10. That material portion is generally flush with the mask body so as to thereby define panels 17 of the second material within the structure of the mask body. The same flexible material, e.g. an elastomer, that is used to form the sealing formation 20 may be used to form the panels 17. The panels 17 may be formed as part of the same two-shot injection moulding process used to provide the mask body 10 with the sealing formation 20. That is to say, a second shot of the multi-shot moulding process may inject the softer material both about the peripheral edge of the mask body (i.e. to provide the seal 20) and also in to the openings 16 (i.e. to provide the panels 17). The seal 20 and panels 17 may thus be formed at the same time.

The mould and injection points for the second shot of the moulding process may be arranged accordingly as would be understood by the skilled person in this field. In achieving the desired multi-shot moulding process, the rigid mask body 10 may be formed in a first mould and transferred to a second mould shaped to define the seal 20 and panels 17. Alternatively the mask body may be formed in a single mould having multiple cavities, whereby the softer material is injected into the cavities defining the seal 20 and panels 17 after the mask body material, i.e. whilst the mask body portion 10 is cooling.

In other embodiments, the seal 20 and panels 17 may be formed at different stages or shots of a multi-shot moulding process. Additionally or alternatively, the panels and seal may be formed of different materials.

In the examples of FIGS. 1 and 2, the panels 17 are elongate in nature. The panels 17 are disposed between the sealing formation 20 and the front wall 13 of the mouth portion 11, and may extend upwards or towards the first end 12a of the mask body such that they also extend into the nose portion 12 of the mask body 10.

The panels 17 are generally planar or smoothly contoured to follow the profile of the mask body. However in other examples, it is possible that the panels could themselves by contoured, for example in a wavy or concertina pattern.

The panels 17 extend only part way along the openings 16 as can be seen clearly in FIG. 2. Accordingly a portion of the opening 16 in the mask body is exposed to define an outlet or exhalation opening 18. The exhalation opening 18 is preferably provided in the lower portion of the elongate opening 16, i.e. in the vicinity of the mouth portion, typically on either side of the front wall 13. The openings 18 may be any or any combination of elongate, quadrilateral or generally rectangular in plan.

In this embodiment, the material of the panels (i.e. the softer material) also extends about the exhalation opening 18 so as to form a border or edge formation about the opening. This provides a softer edge or trim about the opening.

The sealing formation 20 is a unitary flange member that is bonded to, and extends from, the peripheral edge of the mask body 10. The sealing formation 20 may pass substantially around the entire periphery of the mask body 10 and may comprise an inwardly depending lip portion 21, which extends into the opening defined by the edge of the mask body 10. The sealing portion 20 may have discontinuities therein in the form of slits 22 which allow the seal 20 to deform about the different contour portions of a wearer's face. Such slits 22 are provided in the region of the apex and also in the lip portion where it is intended to contact a wearer's cheeks and/or lower jaw in use.

The mask body 10 further comprises an inlet port 19 in the mask body for connection to a supply of an inhalation gas, such as oxygen. The inlet port 19 comprises an opening in the lower wall of the nose portion 12, and a tubular connector 21 that extends outwardly/downwardly away from the mask body 10 into the space in front of the mouth portion 11. The free end of the connector 21 is thus disposed outside of the mask body 10 in front of the mouth portion 11. In use, a supply of an inhalation gas is connected to the tubular connector 21 of the inlet port 19 via a supply tube so as to supply the inhalation gas to the cavity of the respiratory mask and hence the airways of the patient.

The elastomeric nature of the sealing formation 20 enables an effective seal to be formed between the contact surface of the respiratory mask and the face of the patient. However it will be appreciated that the mask may adopt different sealing formations about its peripheral edge in line with other conventional mask designs. Furthermore it is possible that the provision of the second, more-flexible sealing material about the periphery of the mask body may be omitted altogether in the event that the seal quality is of little consequence to the mask provider.

In this embodiment the exhalation openings 18 are simple apertures in the wall of the mask body 10 that allow exhaled gases to exit the cavity of the respiratory mask with little flow resistance. The exhalation openings 18 may be elongate in form. A generally vertically aligned exhalation opening 18 is provided on either side of the font face of the mouth portion 11 (i.e. on side walls 14). The exhalation openings 18 are located at the lower end of the flexible panels 17 but could otherwise be located towards an upper end of the panels 17 or else part-way along a panel 17, thereby dividing the panel into two parts, one on either side of the exhalation opening. It will be appreciated that other shapes, configurations and orientations of exhalation openings 18 are possible. In some embodiments, the exhalation openings 18 may comprise a simple valve structure.

The mask body has a pair of outwardly extending flange formations 23 on either side of the respiratory mask which are arranged to receive an elastic strap in use. Each flange is located adjacent the peripheral edge of the mask body and has an aperture, to which an elastic strap (not shown in the Figures) is attached, in use. The elastic strap extends between the flanges 23, and fits around the patient's head when the respiratory mask is fitted to the patient. In use, the strap is adjusted so that the respiratory mask is urged against the face of the patient with an appropriate force to ensure that an effective seal is formed between the periphery of the respiratory mask and the wearer's face, without causing excessive discomfort for the wearer.

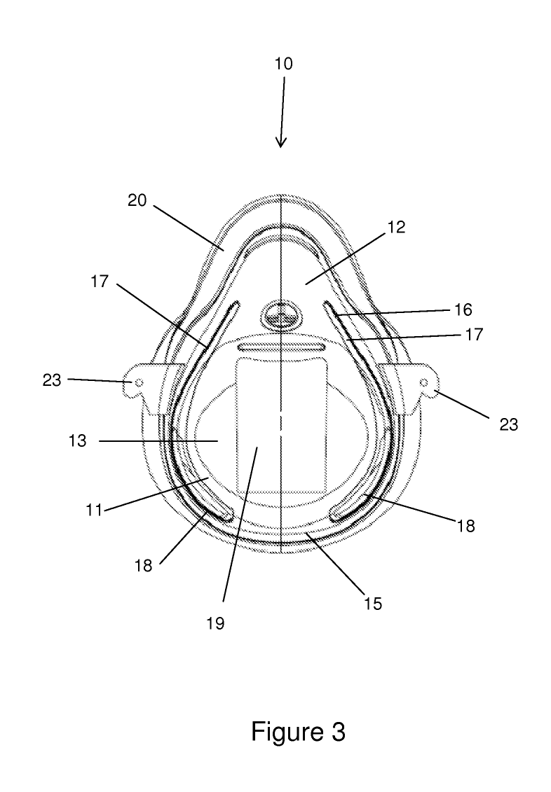

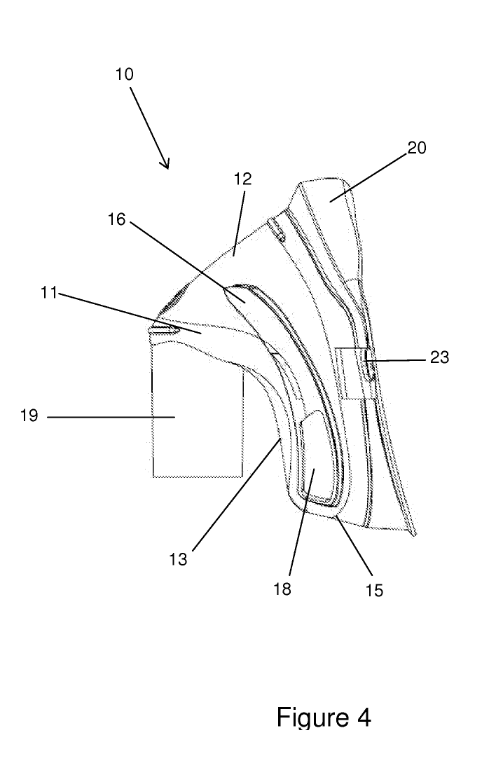

Whilst the above description refers to a mask type typically used for supply of oxygen to a patient, the invention may also be applied to other patient therapy mask types, such as an aerosol mask, as shown in FIGS. 3 and 4. In those figures, like parts have been given like numerals and will not be described again for brevity. The mask of FIGS. 3 and 4 differs from that of FIGS. 1 and 2 only in relative dimensions to accommodate a larger inlet port 19 and connector 21 for delivery of aerosol medication to the patient's airway.

In other embodiments, a reservoir bag of conventional type may be provided in communication with the mask inlet so as to provide a so-called high-concentration mask. Inhalation gas may collect in the reservoir bag over a period of time whereby a patient inhales supplied gas collected within the reservoir bag so as to satisfy a greater volume of inhaled gas than is provided instantaneously by flow of gas to the patient.

In further examples of the invention, the mask may be provided with an exhalation valve in place of the simple exhalation openings described above. The rigid material of the mask body may be shaped so as to define a valve seat within the mask body. A valve member may be held, for example on a spigot or stem formation, over the valve opening so as to block the flow of ambient gas into the mask cavity during inspiration, whilst opening to allow expired gas to escape the mask during expiration. The valve member may pivot, tilt or otherwise deform or be displaced between its open and closed conditions.

A valve arrangement of the type described in the applicant's co-pending International Patent Application No. PCT/GB2012/050676 (published as WO 2012/150441) may be used, the entire contents of which patent application are hereby incorporated by reference. The valve may be provided at one end of a panel as hereinbefore described or else may be provided at a location in the mask body remote from the, or each, panel.

1. A respiratory mask for delivering inspiratory gas to a wearer, the respiratory mask comprising:
a mask body of generally concave shape and having a peripheral edge, the mask body being formed of a first material and adapted to provide a cavity in use about the mouth and nose of a wearer such that the inhalation gas can be inhaled by the patient from the cavity,
wherein the first material of the mask body has at least one opening therein, said opening being at least partially filled with a second material which is softer than the first material so as to define a flexible panel of the second material within the mask body.

2. A respiratory mask according to claim 1, wherein the panel is spaced from the peripheral edge of the mask body by a region of the first material.

3. A respiratory mask according to claim 1, wherein the mask body has a resilient seal formation depending from at least a portion of the peripheral edge, the seal formation arranged to contact and seal against the face of a wearer.

4. A respiratory mask according to claim 3, wherein the resilient seal formation is formed of the second material.

5. A respiratory mask according to claim 1, wherein the mask has first and second ends, the mask body being shaped to accommodate the nose of a wearer towards the first end of the mask and to accommodate the mouth of a wearer towards the second end of the mask, wherein the panel extends part way between the first and second ends.

6. A respiratory mask according to claim 5, wherein the panel extends from the nose accommodating portion to the mouth accommodating portion of the mask body.

7. A respiratory mask according to claim 1, wherein the panel is elongate in form.

8. A respiratory mask according to claim 1, wherein the second material is elastomeric.

9. A respiratory mask according to claim 1, wherein the second material is a moulded material, thereby forming a bond between the first and second materials at the interface therebetween.

10. A respiratory mask according to claim 1, comprising a plurality of said openings and panels, at least one panel being provided on opposing lateral side wall portions of the mask body.

11. A respiratory mask according to claim 1, wherein the panel only partially fills the opening in the first material such that the remainder of the opening defines an exhalation opening in the mask body.

12. A respiratory mask according to claim 1, wherein the panel of the second material portion is substantially flush with the first material portion of the mask body.

13. A respiratory mask according to claim 1, wherein the mask comprises a patient therapy mask having an inlet port that is engageable with a supply of inhalation gas and in communication with the cavity.

14. A respiratory mask according to claim 1, wherein the mask body and panels are formed by a multi-shot moulding process.

15. A respiratory mask for delivering inspiratory gas to a patient, the respiratory mask comprising:
a mask body of generally concave shape and having a peripheral edge, the mask body being formed of a first material and adapted to provide a cavity in use about the mouth and nose of a wearer such that the inhalation gas can be inhaled by the patient from the cavity, the cavity having a mouth portion and a nose portion;
an inlet port that is engageable with a supply of inhalation gas and in communication with the cavity;
a resilient seal formation depending from at least a portion of the peripheral edge; and
a plurality of panels formed of a second material that is more compliant than the rest of the mask body, said panels arranged to permit lateral deflection of the mask body in use to conform to a patient's face.

16. A method of manufacturing a respiratory mask comprising:
moulding a mask body of generally concave shape from a first material in liquid phase at elevated temperature, the mask body having a peripheral edge and being of generally concave shape to define a cavity over the nose and mouth of a wearer in use, wherein the mask body has at least one opening spaced from the peripheral edge;
moulding a second material in liquid phase at elevated temperature within the opening in the mask body so that the second material at least partially fills said opening,
wherein the first material cools to form a generally rigid mask body and the second material cools to form a more flexible panel within the mask body.

17. A method according to claim 16, wherein the first and second material portions are formed as separate stages of a multi-shot injection moulding process.

18. A method according to claim 16, wherein the second material is overmoulded onto the first material of the mask body.
